(19) United States
(12) Patent Application Publication
(10) Pub. No.: US 2025/0281050 A1
(43) Pub. Date: Sep. 11, 2025

(54) Remote Sensing of Bio-Signals

(71) Applicant: Heriot-Watt University, Edinburgh (GB)

(72) Inventors: Souheil Ben Smida, Edinburgh (GB); Panagiota Kontou, Edinburgh (GB); Dimitrios Anagnostou, Edinburgh (GB)

(73) Assignee: Heriot-Watt University, Edinburgh, JJ (GB)

(21) Appl. No.: 18/862,114

(22) PCT Filed: May 4, 2023
(86) PCT No.: PCT/GB2023/051179

§ 371 Date: Oct. 31, 2024

(30) Foreign Application Priority Data
May 5, 2022 (GR) 20220100381
May 17, 2022 (GB) 2207198.9

(51) Int. Cl. A61B 5/0205; A61B 5/00; A61B 5/024

(52) U.S. Cl. CPC A61B 5/0205; A61B 5/024; A61B 5/7207; A61B 5/7225; A61B 5/7264; A61B 5/7271

(57) ABSTRACT

An apparatus (10) for performing a remote measurement of a bio-signal of a subject, the device comprising: at least one radar signal transmitter (14) configured to transmit at least a first radar signal at a first carrier frequency and a second radar signal at a second carrier frequency; at least one signal receiver (16) configured to receive at least one radar return signal; detection circuitry (18) coupled to the signal receiver, wherein the detection circuitry (18) is configured to produce at least one first detection signal for the first carrier frequency and at least one second detection signal for the second carrier frequency in response to receiving the at least one radar return signal at the signal receiver (16), wherein the first carrier frequency and the second carrier frequencies are such that the at least one first and/or the at least one second detection signals comprise a contribution from the bio-signal and a contribution from a further bio-signal and/or a background; and signal combining circuitry (20) configured to perform a signal combining process with the at least one first detection signal and the at least one second detection signal to produce at least one combined signal comprising an at least reduced contribution from the further bio-signal and/or background.

### FIELD

The present invention relates to an apparatus and method for radar-based remote sensing of bio-signals, for example, respiration and heartbeat bio-signals.

### BACKGROUND

Traditional vital sign monitoring systems require patients to wear devices and on-body sensors with wires or otherwise attached to electrodes. These systems may be intrusive, may limit mobility, and their setup may be time consuming. Furthermore, some patients may repeatedly forget or decline to ‘wear’ their sensors due to comfort issues or for other reasons, such as adverse skin reactions, or Alzheimer/Dementia condition.

It is known to sense bio-signals remotely. Remote vital sign monitoring systems that use cameras may cause privacy concerns for the subject being monitored. Additionally, systems that use thermo sensors, can detect remotely respiration only. Other known approaches to remote sensing of bio-signals include radar-based methods and/or methods that use Wi-Fi signals.

Known radar-based approaches include single frequency radar and frequency modulated continuous wave (FMCW) based radar detection. Some known radar-based systems can detect respiration and heartbeat signals simultaneously at the expense of very limited heartbeat accuracy. In such systems, the respiration signal has a larger dynamic range than the heartbeat one which may result in poor resolution detection of the latter.

### SUMMARY

In accordance with a first aspect, there is provided an apparatus for performing a remote measurement of a bio-signal of a subject, the device comprising at least one radar signal transmitter configured to transmit at least a first radar signal at a first carrier frequency and a second radar signal at a second carrier frequency; at least one signal receiver configured to receive at least one radar return signal; detection circuitry coupled to the signal receiver, wherein the detection circuitry is configured to produce at least one first detection signal for the first carrier frequency and at least one second detection signal for the second carrier frequency in response to receiving the at least one radar return signal at the signal receiver, wherein the first carrier frequency and the second carrier frequencies are such that the at least one first and/or the at least one second detection signals comprise a contribution from the bio-signal and a contribution from a further bio-signal and/or a background; and signal combining circuitry configured to perform a signal combining process with the at least one first detection signal and the at least one second detection signal to produce at least one combined signal comprising an at least reduced contribution from the further bio-signal and/or background.

The bio-signal may comprise or represent a heartbeat signal and wherein the further bio-signal and/or wherein the background comprises at least one of: a respiration signal and/or a movement artefact. The first bio-signal may be a heartbeat signal and the further bio-signal may be a respiration signal. The first carrier frequency may be 2.45 GHZ and the second carrier frequency may be 5.8 GHZ.

The first carrier frequency and the second carrier frequencies may be selected and/or have a value such that the at least one first and/or the at least one second detection signals comprise a contribution from the bio-signal and a contribution from a further bio-signal and/or a background. The at least one first detection signal and the at least one second detection signal may comprise complex signals and/or orthogonal signals.

The at least one radar signal transmitter may be coupled to a radar signal generator.

The first carrier frequency and the second carrier frequency may each have a value such that the at least one first detection signal and the at least one second detection signal comprise a contribution from the bio-signal and a contribution from a further bio-signal and/or a background. The first carrier frequency and the second carrier frequencies may have a value such that each of the at least one first detection signal and each of the at least one second detection signals comprise a contribution from the bio-signal and a contribution from a further bio-signal and/or a background.

The value of the first and/or second carrier frequencies may be selected such that the at least one return radar signal comprises or is representative of information associated with the bio-signal and the further bio-signal and/or the background. The value of the first and/or second carrier frequencies may be selected such that the at least one first detection signal and/or the at least one second detection signal is representative of information associated with the bio-signal and the further bio-signal and/or the background.

The signal combining process may comprise processing the at least one first and/or second detection signals to cancel or at least offset their respective contributions from the further bio-signal and/or background. The signal combining process may comprise combining the at least one first and/or second detection signals to cancel or at least offset their respective contributions from the further bio-signal and/or background.

The apparatus as claimed in any preceding claim, wherein the at least one first detection signal comprises two orthogonal detection signals detected for the first carrier frequency and wherein the at least one second detection signal comprises two orthogonal detection signals detected for the second carrier frequency.

The at least one first detection signal may comprise a first in-phase signal and a first quadrature signal and the at least one second detection signal may comprise a second in-phase signal and a second quadrature signal. The signal combining process may comprise combining the first and second in-phase signals and combining the first and second quadrature signals to produce a first in-phase combined signal and a second quadrature combined signal.

The signal combining process may comprise performing a first signal combining process on the first and the second in-phase signals to produce a first combined signal and a second signal combining process on the first and second quadrature signals to produce a second combined signal. The signal combining process may comprise performing a further signal combining process on the first and second combined signals. The first signal combining process may comprise processing the first in-phase signal and the second in-phase signal to cancel or at least offset their respective contributions from the further bio-signal and/or background. The second signal combining process may comprise processing the first quadrature signal and the second quadrature signal to cancel or at least offset their respective contributions from the further bio-signal and/or background.

The signal combining process may be performed in the analogue domain.

The signal combining process may comprise determining a relationship and/or a difference between the at least one first and/or the at least one second detection signals and processing said signals to compensate for said relationship and/or difference.

The signal combining process may comprise performing a scaling and/or a shifting process on the at least one first and/or the at least one second detection signals.

The apparatus may further comprise amplification and/or digitization circuitry for performing an amplification and/or digitisation process on the at least one combined signal.

The apparatus may comprise a passive reflectometer circuit. The passive reflectometer circuit may comprise a 5-port reflectometer.

The 5-port circuit may allow integration and design of a dual-frequency prototype. The passive reflectometer circuit may be coupled to the at least one radar signal transmitter, the signal receiver and a signal generator.

The reflectometer circuit may comprise at least three power detectors. The at least one first and the at least one second detection may be constructed using measurement of power using said three power detectors.

The detection circuitry may comprises a first IQ demodulation module configured to perform a first demodulation or decoding process to produce in-phase and quadrature signals for the first frequency and a second IQ demodulation module configured to perform a second demodulation process to produce in-phase and quadrature signals for the second frequency.

The detection circuitry may comprise two detection chains for each carrier frequency. The detection circuitry may comprise two detection chains for the first carrier frequency and two detection chains for the second carrier frequency.

The first and second carrier frequencies may be selected such that one of the first and second frequencies is lower than the other, higher frequency, wherein the lower frequency is selected to allow sensing of larger signal variations over time and wherein the higher frequency is selected to allow sensing of both larger signal variations and smaller signal variations.

The larger signal variation may correspond to the further bio-signal and/or the background. The smaller signal variations may correspond to the bio-signal being sensed.

The at least one radar signal transmitter may be configured to transmit a further radar signal at a third carrier frequency. The detection circuitry may be further configured to produce at least one third detection signal for the third carrier frequency and the signal combining circuitry may be configured to use the third detection signal.

The at least one radar signal transmitter may be configured to transmit a plurality of radar signals at a corresponding plurality of carrier frequencies. The signal combining circuitry may be configured to perform a signal combining process using the corresponding plurality of detection signals.

The signal processing circuitry may be configured to process the at least one combined signal or a signal derived from the at least one combined signal to determine a heart condition and/or a health condition and/or a health status of a subject.

The processing circuitry may be further configured to apply a pre-determined model to the at least one combined signal and/or a signal derived from the at least one combined signal. The processing circuitry may be further configured to apply a pre-determined model to the at least one combined signal and/or a signal derived from the at least one combined signal to determine a heart condition and/or a health condition and/or a health status of a subject.

The at least one combined signal may comprise first and second combined signals, wherein the first and second combined signals are orthogonal and wherein the at least one signal processing circuitry is configured to apply a trained model to the orthogonal signals or signals derived from the orthogonal signal to determine a heart condition and/or a health condition and/or a health status of a subject.

The apparatus may comprise a further sensor configured to sense a background electromagnetic field corresponding to a communication network. The signal combining process may further comprise reducing the contribution to the combined signal from the sensed background electromagnetic field based on the output from the further sensor.

The background electromagnetic field may comprise an electromagnetic-based communication network having a frequency substantially close to the first and/or second carrier frequencies. The electromagnetic-based communication network may comprise, for example, a LAN, WiFi, NFC, or Bluetooth network. The communication network may have a frequency range overlapping with the first and/or second carrier frequencies. The communication network may have a frequency or frequency range substantially close to the first and/or second carrier frequencies to cause interference.

The signal combining process may further comprise applying a pre-determined model to at least one of: the at least one first detection signal, the at least one second detection signal, a signal representative of the background electromagnetic field, topological and/or subject information to determine the combined signal.

The model may be a machine learning derived model based on at least one of the following features: features in the time domain; feature in the frequency domain; topological or spatial features; radar features; background features; Doppler shift features; reflection signature features.

The model may comprise at least one of: a convolutional neural-network; a recurrent neural network, for example, a long-short term memory network.

The signal transmitter may comprise a Doppler-radar based signal transmitter.

The signal transmitter may be configured to transmit a first electromagnetic wave at the first carrier frequency and a second electromagnetic wave at the second carrier frequency. The signal transmitter may comprise a first signal transmitter configured to transmit a first electromagnetic wave at the first carrier frequency and a second signal transmitter configured to transmit a second electromagnetic wave at the second carrier frequency.

The first carrier frequency may be in the range 300 MHz to 300 GHZ, optionally between 1 GHz and 10 GHz, optionally substantially 2.45 GHZ. The second carrier frequency may be in the range 300 MHz to 300 GHZ, optionally between 1 GHz and 10 GHZ, optionally substantially 5.8 GHZ. The first carrier frequency may be 2.45 GHZ and the second carrier frequency may be 5.8 GHz.

The signal receiver and signal transmitter may form part of a dual-band transceiver.

The signal receiver may comprise at least one antenna. The signal transmitter may comprise at least one signal antenna.

In accordance with a second aspect, which may be provided independently, there is provided a method comprising: transmitting at least one radar signal at a first carrier frequency and a second radar signal at a second carrier frequency; receiving at least one radar return signals; produce at least one first detection signal for the first carrier frequency and at least one second detection signal for the second carrier frequency in response to receiving the at least one radar return signals, wherein the first carrier frequency and the second carrier frequencies are such that the at least one first and/or the at least one second detection signals comprise a contribution from the bio-signal and a contribution from a further bio-signal and/or a background; perform a signal combining process with the at least one first detection signal and the at least one second detection signal to produce at least one combined signal comprising an at least reduced contribution from the further bio-signal and/or background.

The method may further comprise using the at least one combined signal to determine a value of a parameter associated with the bio-signal and/or the further bio-signal and/or to classify a health condition.

The method may further comprise applying a model to the at least one combined signal or data derived from the at least one combined signal to obtain the value of the parameter associated with the bio-signal and/or the further bio-signal and/or the classify a health condition.

In accordance with a third aspect, which may be provided independently, there is provided a method comprising: processing combined signal data representative of at least one combined signal to obtain bio-signal information associated with the bio-signal. The processing of the data may comprise applying a machine learning model or other mathematical processing steps to the data. The processing of the data may comprise applying a trained artificial neural network model to the combined signal data. The bio-signal information may comprise a parameter associated with the bio-signal or other health information.

The combined signal data may be data representative or derived of the at least one combined signal using the method of the second aspect. The combined signal data may be digitized IQ data.

In accordance with a fourth aspect, which may be provided independently, there is provided an apparatus comprising processing circuitry configured to perform the method of the third aspect.

Features in one aspect may be provided as features in any other aspect as appropriate. For example, features of the apparatus may be provided as features of a method and vice versa. Any feature or features in one aspect may be provided in combination with any suitable feature or features in any other aspect.

### BRIEF DESCRIPTION

Various aspects of the invention will now be described by way of example only, and with reference to the accompanying drawings, of which:

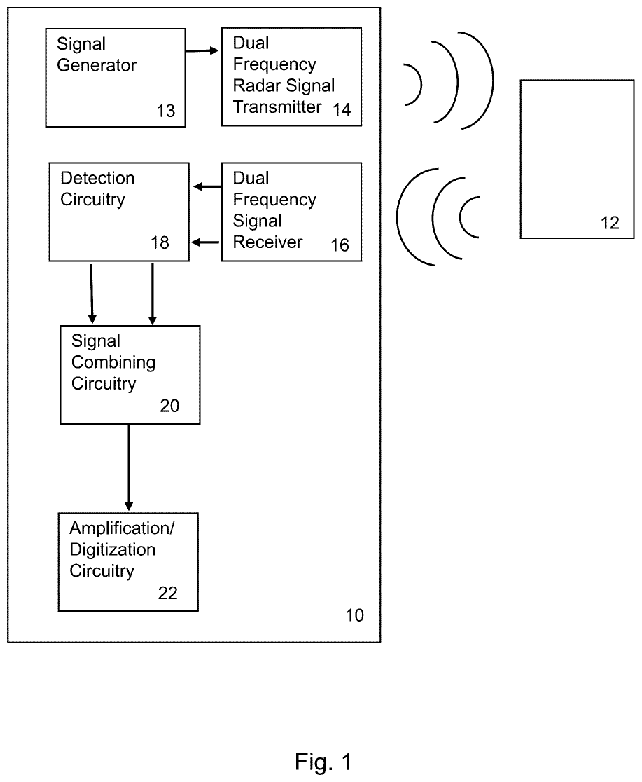

FIG. 1 is a schematic diagram of an apparatus for performing a radar-based remote measurement of bio-signals, in accordance with an embodiment;

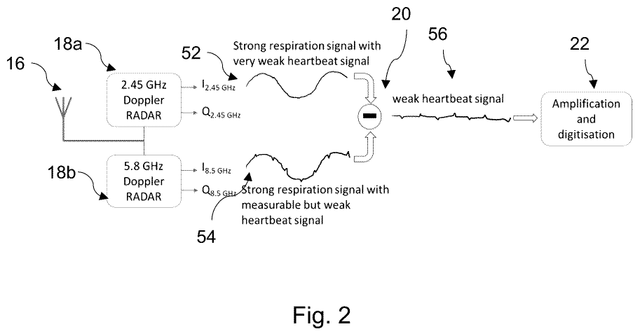

FIG. 2 illustrates a signal combination process, in accordance with embodiments;

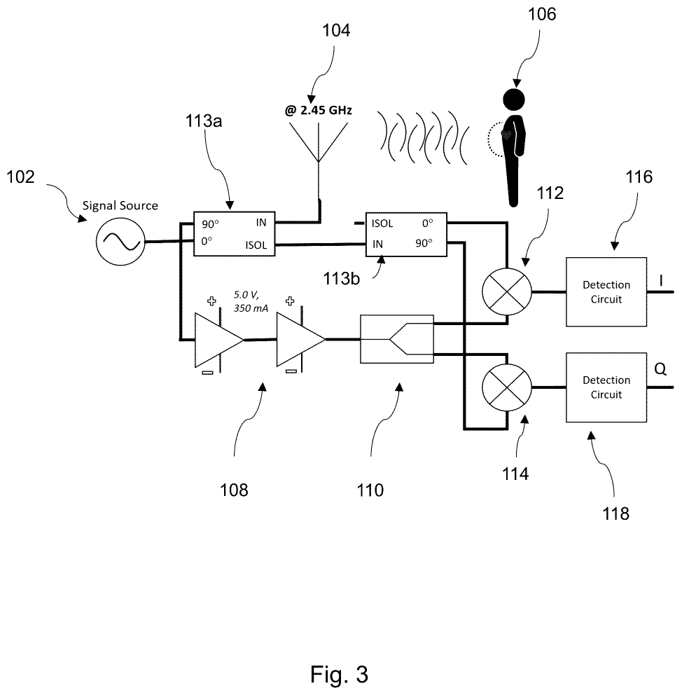

FIG. 3 is a diagram of a radar module for a first frequency;

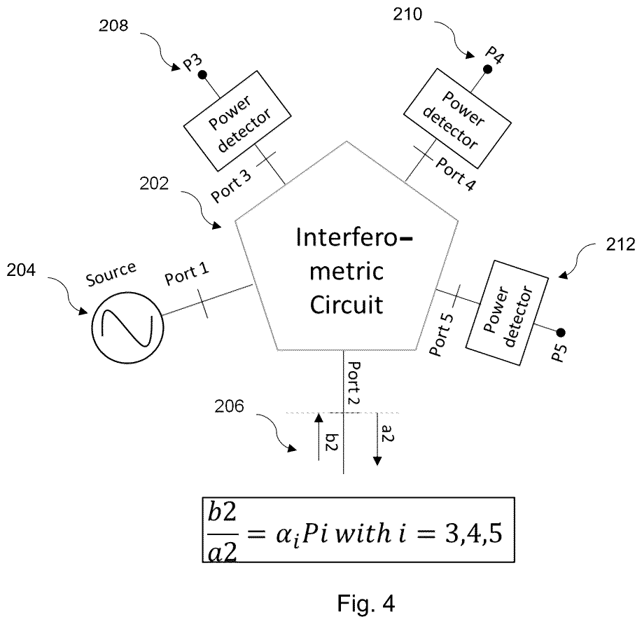

FIG. 4 is a schematic diagram of an interferometric circuit, in accordance with embodiments, and

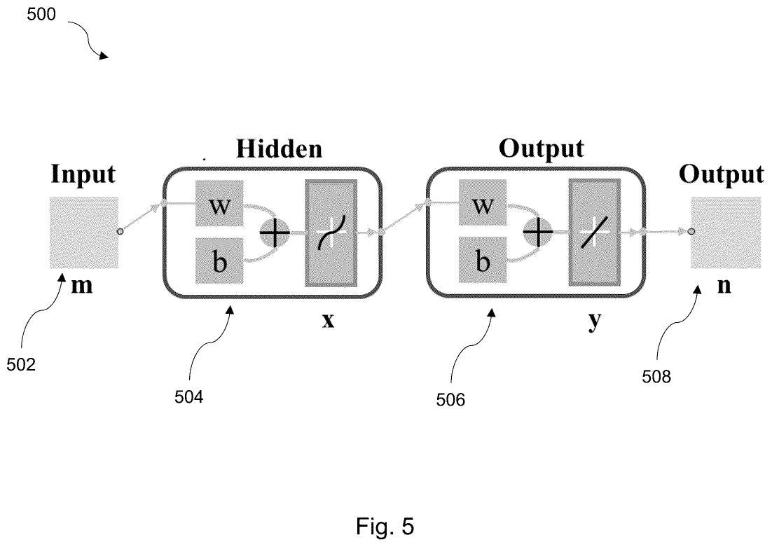

FIG. 5 is a schematic diagram of neural network model.

### DETAILED DESCRIPTION

FIG. 1 is a schematic diagram of an apparatus 10 for radar based remote sensing of bio-signals. The present embodiment uses two different carrier frequencies for radar sensing. It will be understood that, in further embodiments, more than two carrier frequencies may be used.

As described in the following, the apparatus 10 is configured to perform measurement of a bio-signal of interest (a heartbeat signal, in the present embodiment). The apparatus 10 suppresses a second bio-signal and/or background. In the present embodiment, the second bio-signal being suppressed is a respiration measurement. The apparatus 10 thus measures bio-signals at two different ISM bands concurrently (for example 2.45 GHz and 5.8 GHZ) and uses a single dual-frequency transceiver, to correlate the measured respiration signals in order to cancel them out thereby to boost the heartbeat signal to achieve useful signal-to-noise-ratio. By using radar-based measurement of bio-signals, the apparatus is provided remotely from the subject to allow remote sensing. The apparatus may therefore be considered as a contact-less or wireless sensing apparatus.

The present embodiment relates to sensing of a first bio-signal which is a heartbeat signal from the subject 12. The first bio-signal thus corresponds to or is representative of a heartbeat of a subject 12. The present embodiment also suppresses a second bio-signal which is a respiration signal from the subject 12. The second bio-signal thus corresponds to or is representative of a respiration rate of the subject 12. While the present embodiment is related to these bio-signals, it will be understood that the same apparatus may be used and/or modified to sense other bio-signals and/or to suppress other bio-signals and/or background signals. Likewise, while the electromagnetic carrier frequencies described in the present embodiment are 2.45 GHZ and 5.8 GHz, other frequencies can be used in other embodiments. The frequencies used may be selected based on the bio-signals of interest.

The apparatus described may allow an improved heartbeat signal quality that may be exploited for diagnosis purpose, which may be contrasted with monitoring method that provide simpler, heartbeat counting, for example, for health tracking purposes.

Turning to FIG. 1, the apparatus 10 has a signal generator 13 coupled to a dual frequency radar transmitter 14 (also referred to, for brevity as a signal transmitter), a dual frequency radar signal receiver 16 (referred to, for brevity as a signal receiver), detection circuitry 18 and signal combining circuitry 20. For the purpose of the following description, the signal receiver and transmitter are depicted as two separate modules, however, it will be understood that the signal receiver and the transmitter can be provided as a single transceiver, in particular, a dual-band transceiver. The apparatus 10 also has amplification/digitization circuitry 22. However, it will be understood that the amplification/digitization circuitry 22 may be provided separately to the apparatus.

The signal generator 13 is coupled to the radar signal transmitter 14 and together these components are configured to generate and transmit free-space electromagnetic waves that are suitable for performing Doppler radar. In the present embodiment, the radar signal transmitter 14 is configured to transmit a first radar signal at a first carrier frequency and a second radar signal at a second carrier frequency. The first radar signal is an electromagnetic wave at frequency 2.45 GHZ. The second radar signal is an electromagnetic wave at frequency 5.8 GHZ. The radar signal transmitter 14 is configured to transmit the electromagnetic waves towards the subject 12.

The dual frequency signal receiver 16 is configured to receive electromagnetic radar return signals at the first and second frequency. At least some of the radar return signals are reflected or scattered by the subject 12 and thus these return signals carry information about the subject 12. In particular, by appropriate selection of the first and second carrier frequencies, the information carried by the return signals comprises bio-signal information. In the present embodiment, the 5.8 GHz return signal carries information about the bio-signal of interest (the heartbeat signal) and a further bio-signal (the respiration signal). Likewise, the 2.45 GHz return signal carries information about both bio-signals, however, due to the carrier frequency, the contribution to the return signal from the heartbeat is smaller than for the 5.8 GHz return signal.

The signal receiver 16 is coupled to detection circuitry 18. Further detail regarding the specific implementation of the detection circuitry is provided with reference to FIG. 2 and FIG. 3. In the present embodiment, the detection circuitry 18 has two detection modules, one detection module for each Radar frequency. Each detection module comprises a detection chain—a series of hardware components for converting the radar return signal to a detection signal.

For each frequency, the detection module of the detection circuitry is configured to convert the received Radar return signal and perform an in-phase/quadrature (IQ) demodulation process on the return signal to produce an in-phase signal and a quadrature signal. The in-phase and quadrature signals are then combined at the signal combining circuitry 20 to produce one or more detection signals. The in-phase and quadrature signals can be considered as examples of orthogonal signals.

The detection circuitry 18 is coupled to the signal combining circuitry 20. The signal combining circuitry is configured to perform a signal combining process to combine detection signals corresponding to the first carrier frequency and detection signals corresponding to the second carrier frequency. The combined signal has a contribution from the heartbeat signal, however, the combination of detection signals has substantially cancelled the respective respiration contributions from the first and second detection signals.

In the present embodiment, the signal combining circuitry operates as follows. In the present embodiment, for each frequency, each system provides two baseband signals, one in-phase signal (referred to as I) and one quadrature signal (called Q). A signal combining process is then performed on the two in-phase signals from each radar (i.e. the first frequency in-phase signal and the second frequency in-phase signal). The signal combining process is performed, for example, to cancel the respiration contribution between the two signals. A further signal combining process is then performed on the two quadrature signals (i.e. the first frequency quadrature signal and the second frequency quadrature signal). The two quadrature signals are combined to cancel respiration contributions. As a result of the signal combining process, two combined analogue signals are produced: a first combined signal (the in-phase combined signal) and a second combined signal (the quadrature combined signal).

The two combined signals are then passed to the amplification and digitization circuitry 22 to be amplified and then digitized. It will be understood that the signal combining process is performed in the analogue domain. The amplification and digitization process produces digital IQ data. The IQ data may also be referred to as combined signal data.

The IQ data may be representative of a combined signal or waveform and may be further processed using further processing circuitry. For example, as described with reference to, for example, FIG. 5, the processing steps may include applying a pre-determined model or mathematical processing steps to obtain bio-signal information or applying mathematical processing steps. The combined in-phase and combined quadrature signals can be considered to correspond to real and imaginary parts of a measured waveform that contains the bio-signal information.

The signal combining process is depicted in more detail with reference to FIG. 2. FIG. 2 schematically depicts signal receiver 16 and the two detection circuitry modules of detection circuitry 18a, 18b (i.e. 2.45 GHz Doppler radar detection module 18a and 5.8 GHz Doppler radar detection module 18b). First detection circuitry module 18a generates a first in-phase detection signal and a first quadrature detection signal. This pair of signals are represented by first detection signal 52 (however, it will be understood that, in the present embodiment, both an in-phase and a quadrature detection signals are produced). Second detection circuitry module 18b generates a second in-phase detection signal and a second quadrature detection signal. This pair of signals are represented by second detection signal 54 (however, it will be understood that, in the present embodiment, both an in-phase and a quadrature detection signals are produced).

As can be observed in FIG. 2, the first detection signal 52 has a respiration contribution and a very weak heartbeat signal contribution. The second detection signal 54 has a respiration contribution and a weak, but measurable, heartbeat signal contribution. FIG. 2 also schematically depicts the signal combining circuitry 20 that performs the signal combining process to produce the combined signal 56.

As depicted in FIG. 2, first and second detection signals are generated in response to receiving radar return signals at the first and second respective frequencies. The first detection signal which is produced in response to receiving the 2.4 GHz Radar return signal, has a strong respiration signal with a very weak heartbeat signal. The second detection signal which is produced in response to receiving the 5.8 GHz Radar return signal, also has a strong respiration signal with a weak, but measurable heartbeat signal. It will be understood that, in some embodiments, the radar detection signals are generated as part of a signal detection process in which the free-space radar return signals are received and converted into detected signals.

In further detail, the first detection signal can be considered as having a first respiration signal contribution and a first heartbeat signal contribution. The second detection signal can be considered as having a second respiration signal contribution and a second heartbeat signal contribution. The two signals are combined by performing a signal combination process that substantially cancels the first respiration signal contribution (from the first detection signal) and the second respiration signal contribution (from the second detection signal). The resulting combined signal is thus a combination of the first heartbeat contribution and the second heartbeat contribution. While these signals will also offset each other, as the second heartbeat contribution (before or after scaling) is larger than the first heartbeat contribution, the resulting signal will have a weak, but measurable heartbeat signal with substantially no, or at least a reduced, respiration signal contribution.

While FIG. 2 illustrates a cancellation of the respiration contributions, it will be understood that a perfect cancellation may not be possible, and an offset or partial cancellation may be performed. As part of the signal cancelling process, a relationship or a difference between the first and second detection signals may be determined and the combination may take into account such a difference. For example, a scaling function may be applied to one of the first or second signals before subtraction so that the respective respiration signal contributions have substantially equal magnitudes (and thus offsets to a greater degrees). Furthermore, time shifting/correlation operations may be performed to ensure, for example, that features of the detection signals align prior to cancellation.

The proposed apparatus exploits the redundancy of respiration bio-signal measurement provided by two measurement systems operating at two different carrier frequencies. As discussed above, to provide sufficient cancellation, the respiration bio-signal analogue scaling functions that are required can be placed after IQ baseband signal detection. The scaling functions can also be implemented in the RF receiver path via amplifiers and the phase shifters (16 in FIG. 2), or by scaling the Local Oscillator power and phase in the receivers 18a and 18b. Because the bio-signal due to heartbeat is very weak in the 2.45 GHz receiver chain and measurable in the 5.8 GHz chain, the combination of the two chains outputs will result in a still measurable heartbeat signal with much smaller component related to respiration. In practice, only around 40 dB of cancellation of respiration bio-signals would be required, which is achievable with analogue scaling circuits.

In the above-described embodiments, the combining process produces one or more combined signals having a reduced contribution from a further bio-signal and/or a background. In some embodiments, the combining process cancels the contributions from the further bio-signal and/or the background such that the combined signals have substantially no contribution from the further bio-signal and/or the background. In some embodiments, it will be understood that the combining of signals is such that the combined signal is proportional to, for example, on average, the bio-signal of interest. In some embodiments, it will be understood that the combining of signals is such that the combined signal has one or more properties (for example, frequency) corresponding to the bio-signal of interest. The combination of signals may be performed in the time domain, and the combined signal may be such that a Fourier transform (or other appropriate transformation to the frequency domain) would produce a frequency domain signal having a dominant frequency component at the frequency of the bio-signal and a supressed (or zero) frequency component at the frequency of the further bio-signal and/or suppressed (or zero) contributions relating to background/movement.

FIG. 3 depicts an apparatus 100 for detecting and signal processing of a single Radar frequency. It will be understood that, for two Radar frequencies, two instances of this circuitry will be provided, however, for brevity, the apparatus for only a single frequency is described in the following. It will be understood that the output of the apparatus of FIG. 3 is a first (in-phase) detection signal and a second (quadrature) detection signal for the single frequency. The detection signals are combined with the corresponding detection signals of the second apparatus (for the second frequency) that is not shown, in the signal combining circuitry (for example, as described with reference to FIG. 1 and FIG. 2).

In further detail, the apparatus 100 of FIG. 3 has a microwave signal generator 102 (also referred to as a signal source) for generating a microwave signal. The apparatus 100 has a transceiver antenna 104 for transmitting and receiving free-space microwave signals. In the present embodiment, the transceiver antenna 104 is a single antenna, however, it will be understood that, in other embodiments, the transceiver antenna 104 may be replaced by a transmitting antenna and a separate receiving antenna. The apparatus 100 also has an amplifier 108, a signal splitter 110, a first mixer 112 and a second mixer 114, and first and second receiver chains 116 and 118 (also referred to as detection circuits).

The apparatus also has a first directional coupler 113a and a second directional coupler 113b. First directional coupler 113a is configured to direct the generated signal from the source 102 to the transmitting antenna 104 as well as to the input of the second directional coupler 113b. First directional coupler 113a is also configured to direct the received signal by the antenna 104 to the input of the amplifying circuits 108. The signal generated by the source is used in the down-conversion process as a local oscillator. The second directional coupler 113b splits the signal generator signal into two local oscillator signals to be used with mixers 112 and 114.

In use, a microwave generating signal is generated from the signal generator 102 and is transmitted through the transmitting antenna 104 towards a subject 106. The transmitting antenna 104 thus transmits a radar signal at a carrier frequency towards the subject 106. The reflected signal, also referred to as a radar return signal, is received by the same antenna 104. The radar return signal is amplitude and/or phase modulated and is received by antenna 104 and is amplified by the amplifier. The amplified signal is split by splitter 110 into two signals: a first and second signal. The first signal is down converted to a baseband signal through mixing with the transmission signal at mixer 112 to produce the first baseband signal. The second signal is downconverter to a baseband signal through mixing with the transmission signal at mixer 114.

The first of the two baseband signals is provided to the first receiver chain 116 and the first receiver chain 116 produces an in-phase, I, detector signal. The second of the two baseband signals is provided to the second receiver chain 118 and the second receiver chain 118 produces a quadrature, Q, detector signal. For each receiver chain, a DC offset reduction is achieved by a detection circuit which allows the isolation of the DC component. That dc component is then subtracted from the original signal. The result is amplified to improve the dynamic range. The output I and Q signals are then fed into an oscilloscope (not shown) for further data collection and signal-processing. The signal-processing includes conversion of the detector signals into digital IQ data. The digital IQ data may also be referred to as combined signal data.

In the above-described embodiments, a signal combining process is described in which detection signals associated with first and second radar frequencies are combined. As described above, for each carrier frequency in-phase and quadrature signals are detected. In some embodiments, the signal combining process includes combining the in-phase signals for the first and second carrier frequencies to suppress background and/or other bio-signals in the combined signal and further combining the signal combining process includes combining the quadrature signals for the first and second carrier frequencies to suppress background and/or other bio-signals in the combined signal. The combined in-phase and combined quadrature signals can be considered to correspond to real and imaginary parts of a measured waveform that contains the bio-signal information.

FIG. 4 depicts an embodiment with an interferometric circuit arrangement, in accordance with embodiments. In the embodiment of FIG. 4, a dual-frequency five-port reflectometer is used as a dual-band IQ demodulator connected to a dual-band antenna. In further detail, FIG. 4 depicts a five port, passive reflectometer 202. The passive reflectometer is coupled to an electromagnetic source 204, a transceiver antenna 206, a first power detector 208, a second power detector 210, and a third power detector 212.

The electromagnetic source 204 generates electromagnetic radar waves at the first and second frequencies. While only a single source is depicted, it will be understood that in some embodiments, two sources are provided to generate radar waves at each of the frequencies (for example, a first electromagnetic source to generate a radar wave at the first frequency and a second electromagnetic source to generate a radar wave at the second frequency) The generated waves are coupled to the transceiver 204 via the reflectometer. The transceiver thus transmits the radar at both frequencies towards the subject (not pictured) and radar return signals are received by the transceiver. The received radar return signals are coupled, via the reflectometer 202 to the three power detectors. By detecting the power at these three detectors, reconstruction of the detection signals for the first and second radar frequencies can be performed.

It will be understood that in some embodiments, a calibration process is performed on the system. Following the calibration process, a measurement of the five-port receiver gives three outputs. The signal processor combines the three outputs with the calibration results to output I and Q. Known calibration techniques may be used. An example calibration method relates the detected voltages to the reflected radar signal. An example of a known calibration method is found, for example, at “Wide-band RF receiver using the “five-port” technology” by Neveux et al.

The five-port receiver gives three voltages, the combination of which provides I and Q signals. The implementation uses the five-port as it is much easier to manufacture, calibrate and obtain accurate results. The three detected voltages from the five-port can be processed using a network of op-amps to provide analogues I and Q signals. An example of a network of operational amplifiers used to provide analogue I and Q signals may be found, for example, in “Performance of 2-3.6 GHZ Five-Port/Three-Phase Demodulators with Baseband Analog Regeneration Circuit in Direct-Conversion Receivers”, by Abdou et. al.

While a standard IQ demodulator may be used in place of the five-port receiver, it was found that five-port receiver may, in certain circumstances, provide advantages such as ease of manufacture, calibration and improvement in accuracy of results.

This embodiment may allow improved heartbeat signal quality (that may outperform available consumer electronic products in terms of quality of detected bio-signals cost-effectively) that could be exploited for diagnosis purpose in contrast to simple heartbeat counting and monitoring that can be performed currently.

As described above, an apparatus and methods of obtaining IQ data is described. As described above, digital IQ data is generated using, for example, the apparatus of FIG. 1, 2, 3 or 4. A description of signal processing of the generated digital IQ data, in accordance with an embodiment, is provided in the following. As described in the following, one or more signal processing steps may be applied to the digital IQ data to obtain bio-signal information. As describe in the following, the bio-signal information may comprise a parameter associated with the bio-signal or, for example, other health information. The signal processing steps can include applying mathematical processing steps, for example, FFT or applying machine-learning derived processing steps, such as applying an artificial neural network to the IQ data.

The digitised IQ data is obtained in the time domain. Firstly, the digitised signal is transformed from the time domain to the frequency domain. In the present embodiment, the signal transformation is a Fast Fourier Transform (FFT). It will be understood that if no signal combination process is performed (i.e. no signal cancellation) two main peaks of the FFT are observed: a first peak corresponding to a respiration frequency and a second peak that is much lower in magnitude corresponding to the heartbeat frequency.

When the signal combination is applied, as described above, the respiration is substantially cancelled and only the heartbeat echo frequency is present in the FFT.

Using the combined data, a suitable algorithm, for example, a peak finding algorithm, can be used to identify the heartbeat rate. However, a filtering of the heartbeat frequency content and an inverse FFT leads to a time domain waveform that represents the mechanical movement of the heart. The combination of, for example, FFT, filtering followed by inverse FFT may be useful to remove band noise. It will be understood that the signal processing steps, such as combining the I and Q data, applying FFT and IFFT, are performed after the signal is converted to a digital signal.

In some embodiments a pre-determined model (a model trained using machine learning derived techniques) is used and applied to the obtained combined data. In some embodiments, the trained model is applied to digital IQ data or data derived from said IQ data. In particular, the cleaned heartbeat waveform constructed using the signal processing techniques as described above is provided as an input to a machine learning model to determine if the waveform corresponds to a heart condition. In particular, the model may identify one or more features in the waveform that represent anomalous heart behaviour and/or heart conditions. Such heart conditions may include, for example, COPD, and arrythmias such as Brugada and others.

FIG. 5 is a schematic diagram of a trained model 500, in accordance with embodiments. The model 500 is a neural network having an input 502, a hidden layer 504, an output layer and an output 506. Each layer has one or more nodes. The connections between nodes of one layer and nodes each have an associated weight. The trained neural network weights therefore relate the input to the output. As the training or learning process is performed, the weights are iteratively adjusted. The neural network model may be considered as a series of equations that are produced using neural network modelling to allow prediction of a parameter associated with the input waveform or other classification.

The training is performed over time to refine the weights. In some embodiments, the input layer is a set of vectors or other data structure representing the waveform data and the method further includes the step of constructing the set of vectors for use in the model. In some embodiments, the model is a series of equations that allow a prediction of the output based on the input waveform. In such embodiments, the training of the model may include producing the series of equations and the application of the model includes applying the equations. In some embodiments, the output layer is a parameter associated with the input waveform (for example, a value of a parameter associated with a first or second bio-signal).

As part of the training process, training data is obtained, and a machine learning process is performed to determine optimal values for model parameters. In the present embodiment, a pre-processing step is first performed on the training data to treat noise and outliers (extreme values that introduce biases in the statistical estimates). In more detail, before feeding the data into the neural network, normalisation, time domain shifting or phase synchronisation, detrending and manipulation of sampling rates can be performed. These are examples and other pre-processing methods may be applied.

The trained model provides a mathematical relationship between a mathematical representation of the input and a mathematical representation of the output. As part of the training, the training data may include both measured and digitally constructed data. The measured data comes from healthy and unhealthy individuals. The digitally constructed data is a version of the measured data that is extrapolated to more case scenarios. As a non-limiting example, a measured data input could be an unhealthy individual with a first heart condition and a heartbeat rate but with different heartbeat. In some embodiments, synthetic data is generated. The synthetic data is generated as sinusoidal functions with frequencies selected to match what may be expected from measurements.

The input nodes are combined in different ways to generate new features which are then used to predict the output. The weights (w) of the hidden layer are the connection between the nodes of the input and the neurons of the hidden layer. The hidden layer is taking the product of the input times the w and it is summing all those terms plus the bias (b) then it feeds that to an activation function. All the neurons of the hidden layer are connected with some weights to the output. The output layer predicts the output of the model. A training process is performed to train the model using training data. As part of the training process, initial weights (w) and biases (b) are generated and then optimized. During the training process, the model learns from the training data at this stage. A validation process is also performed using validation data. The validation data set is used to check the model at each step.

The architecture of the model is optimized as well as other parameters are tuned to get the lowest possible error. The test dataset can be used to assess how well the model is performing on any unknown data and was not biased to work for the validation set.

In a non-limiting example, two hidden layers, with 30 neurons in the first hidden layer and 2 neurons in the second hidden layer and 400 input nodes (a waveform). The input nodes correspond to the input waveform. The training data is divided into 72% data for training set, 18% for validation and 10% for test set. In this embodiment, the output is a single node corresponding to a single value, the frequency of the input waveform. These parameters are provided as a non-limiting example and it will be understood that other sets of parameters may be used.

The input to the neural network includes the generated combined IQ data (the amplified and digitized data obtained, as described above) or a quantity or representation related to the IQ data that is representative of the information obtained. In some embodiments, the input is in the form of a complex function I+iQ or corresponding vector representation. In some embodiments, the input is a quantity related to this complex number, for example, the phase of the complex number. It will be understood that the machine learning network is trained to receive information relating to both the orthogonal, I and Q signals. A single network may not be sufficient to observe any features in the event that one of the I and Q signal comprises all derived information and the other of the I and Q signals does not contain sufficient information. The I or Q signals not having enough information may be due to the position of the subject. The combination of I and Q may therefore be provided as an input to the neural network.

In general, the trained model outputs bio-signal information, which may be, for example, a parameter associated with the measured bio-signal (the bio-signal that is not suppressed or cancelled) or other health information. In some embodiments, the trained model outputs a frequency associated with respiration (for example, a breathing rate or respiration rate). In other embodiments, the output of the model is a heartbeat frequency. In some embodiments, the output is a rate or other desired parameter associated with the bio-signal of interest. The output of the neural network may include, for example, heart condition and the heartbeat rate. In further embodiments, the output of the model is health information, for example, a heart or health condition or health status. In some embodiments, a time domain waveform is provided as an input to a trained neural network. The time domain waveform may be represented as a suitable data structure, for example, vector, array or matrix.

The output of the trained network may vary depending on applications. In some embodiments, the output is a parameter associated with a bio-signal, for example, a heartbeat count, heart rate, or breathing rate. In further embodiments, the output relates to health information, for example, a label corresponding to a range of different heart conditions that are detected by processing the input.

It will be understood that the neural network is trained using training data comprising a set of heartbeat time domain waveforms that are obtained from several cases including both waveforms representative of healthy conditions and waveforms representative of hearts that have certain heart conditions. In some embodiments, the machine learning model detects heart conditions that are present in the way the heart moves in contrast to the classic heart condition investigation with ECG which is an electrical signal of the nerve activity of the heart muscles. In some embodiments, the output from the model is a classification of a heart condition and a heart rate value.

In such embodiments, machine learning derived models are utilized and used to process detected signals. Signal processing steps, such as combining the I and Q data, applying FFT and IFFT are performed in the digital domain. Signal processing algorithms are implemented (such as machine learning (ML), and/or deep learning (DL)) by importing the data as vectors or multidimensional matrices and processing them in time and/or frequency domains to reduce noise further, increase resolution, estimate heartbeat and respiration rates as well as highlighting potential heart conditions.

The model used in the present embodiment is an artificial neural network (ANN). In particular, the network could be a Long-Short Term Memory (LSTM) network or another type of recurrent neural networks (RNNs). These RNNs could integrate the temporal dimension to model both short and long-term dependencies in the data.

In particular, in some embodiments, the above-described algorithms may be extended to integrate data from multiple heterogeneous sources (including radar, Wi-Fi) and exploit a rich set of temporal, frequency and topological features to train and test classifiers based on micro-Doppler and micro-reflection signatures. In contrast to known methods, radar and Wi-Fi target classification and machine learning can be combined.

The combination or fusion of these signals originating from heterogeneous sources may increase the accuracy of the sensed bio-signals by using information from one signal to inform the output of the other signal and vice versa. For example, a Wi-Fi signal may be used to enhance the accuracy of the radar sensing at larger distances. Alternatively, the radar signal may be used to enhance the accuracy of the Wi-Fi signal at shorter distances. The order of the signals and of the distances may be reversed. The method could be any known signal processing method such as correlation, and/or algorithms based on Machine Learning (RNNs).

In further embodiments, sensing accuracy may be further improved by combining radar wave information with sensed background electromagnetic information. In particular, in further embodiments, a further sensor may be provided to sense variations in an ambient electromagnetic communication field (for example, an ambient Wi-Fi signal) in the environment.

While the machine learning model is described for detecting heart conditions, in further embodiments, the machine learning model maybe trained to output a health state of a subject. In some embodiments, the model may detect conditions such as sleep apnoea and/or conditions related to, for example, sudden infant death syndrome. In a further embodiment, the system may be configured to process the IQ data to detect movements, for example, gesture recognition or activity recognition.

The above description of specific embodiments is made by way of example only and not for the purposes of limitation.

By way of non-limiting example, in the above-described embodiments, although the proof of concept is performed using the frequencies of 2.45 GHZ and 5.8 GHZ, it will be understood that other frequency pairs can also be used, such as for example: 2.45 GHZ and 24.5 GHZ, 5.8 GHz and 24.5 GHZ, or other pairs. In general, the lower frequency is selected to sense large signal variations (e.g. breathing, position, gestures, etc.) while the higher frequency is selected to sense both the large and the small signal variations (e.g. heartbeat), for further processing. In general, the selection of frequencies uses may be dependent on the bio-signal of interest and the further bio-signal/background to be suppressed. In a further embodiment, a concurrent measurement at three frequencies (triple frequency radar), for example, at 2.45 GHZ, 5.8 GHZ, and 24.5 GHz may further improve sensing accuracy and the signal processing.

Further, the above embodiments describe sensing of bio-signals, such as heartbeat and/or respiration. A bio-signal may be understood, in accordance with embodiments, as a time and/or spatially varying signal that is representing or indicative of a biological event in a subject, for example, a beating heart, breathing and/or movement/contraction of muscle.

It will be clear to the skilled person that modifications of detail may be made within the scope of the invention.

1. An apparatus for performing a remote measurement of a bio-signal of a subject, the device comprising:
at least one radar signal transmitter configured to transmit at least a first radar signal at a first carrier frequency and a second radar signal at a second carrier frequency;
at least one signal receiver configured to receive at least one radar return signal;
detection circuitry coupled to the signal receiver, wherein the detection circuitry is configured to produce at least one first detection signal for the first carrier frequency and at least one second detection signal for the second carrier frequency in response to receiving the at least one radar return signal at the signal receiver, wherein the first carrier frequency and the second carrier frequencies are such that at least one of the at least one first detection signal or the at least one second detection signal comprise a contribution from the bio-signal and a contribution from at least one of a further bio-signal or a background; and
signal combining circuitry configured to perform a signal combining process with the at least one first detection signal and the at least one second detection signal to produce at least one combined signal comprising an at least reduced contribution from at least one of the further bio-signal or the background.

2. The apparatus as claimed in claim 1, wherein the bio-signal comprises a heartbeat signal and wherein at least one of the further bio-signal or the background comprises at least one of: a respiration signal or a movement artefact.

3. The apparatus as claimed in claim 1, wherein the signal combining process comprises processing at least one of the at least one first detection signal or the at least one second detection signal to cancel or at least offset their respective contributions from at least one of the further bio-signal or the background.

4. The apparatus as claimed in claim 1, wherein the at least one first detection signal comprises a first in-phase signal and a first quadrature signal and the at least one second detection signal comprises a second in-phase signal and a second quadrature signal and wherein the signal combining process comprises combining the first and second in-phase signals and combining the first and second quadrature signals to produce a first in-phase combined signal and a second quadrature combined signal.

5. The apparatus as claimed in claim 1, wherein at least one of a), b) or c):
a) the signal combining process is performed in the analogue domain;
b) the signal combining process comprises determining at least one of a relationship or a difference between at least one of the at least one first detection signal or the at least one second detection signal and processing the signals to compensate for the at least one of the relationship or the difference; or
c) the signal combining process comprises performing at least one of a scaling or a shifting process on at least one of the at least one first detection signal or the at least one second detection signal.

6. (canceled)

7. (canceled)

8. The apparatus as claimed in claim 1, further comprising at least one of amplification circuitry or digitization circuitry for performing an amplification process or a digitisation process, respectively, on the at least one combined signal.

9. The apparatus as claimed in claim 1, at least one of: wherein the apparatus comprises a passive reflectometer circuit or wherein the passive reflectometer circuit comprises a 5-port reflectometer.

10. (canceled)

11. The apparatus as claimed in claim 1, wherein the detection circuitry comprises at least one of a) or b):
a) a first IQ demodulation module configured to perform a first demodulation or decoding process to produce in-phase and quadrature signals for the first frequency and a second IQ demodulation module configured to perform a second demodulation process to produce in-phase and quadrature signals for the second frequency; or
b) two detection chains for each carrier frequency.

12. (canceled)

13. The apparatus as claimed in claim 1, wherein the first and second carrier frequencies are selected such that one of the first and second frequencies is lower than the other, higher frequency, wherein the lower frequency is selected to allow sensing of larger signal variations over time and wherein the higher frequency is selected to allow sensing of both larger signal variations and smaller signal variations.

14. The apparatus as claimed in claim 13, wherein the larger signal variation corresponds to at least one of the further bio-signal or the background, and wherein the smaller signal variations correspond to the bio-signal being sensed.

15. The apparatus as claimed in claim 1, wherein at least one of a) or b):
a) the at least one radar signal transmitter is configured to transmit a further radar signal at a third carrier frequency, wherein the detection circuitry is further configured to produce at least one third detection signal for the third carrier frequency and the signal combining circuitry additionally uses the third detection signal; or
b) the at least one radar signal transmitter is configured to transmit a plurality of radar signals at a corresponding plurality of carrier frequencies, and wherein the signal combining circuitry is configured to perform a signal combining process using the corresponding plurality of detection signals.

16. (canceled)

17. The apparatus as claimed in claim 1, further comprising signal processing circuitry configured to process the at least one combined signal or a signal derived from the at least one combined signal to determine at least one of: a heart condition, a health condition, or a health status of a subject.

18. The apparatus as claimed in claim 17, wherein the at least one combined signal comprises first and second combined signals, wherein the first and second combined signals are orthogonal and wherein the at least one signal processing circuitry is configured to apply a trained model to the orthogonal signals or signals derived from the orthogonal signal to determine a heart condition.

19. The apparatus as claimed in claim 1, further comprising a further sensor configured to sense a background electromagnetic field corresponding to a communication network and wherein the signal combining process further comprises reducing the contribution to the combined signal from the sensed background electromagnetic field based on the output from the further sensor.

20. The apparatus as claimed in claim 1, wherein the signal combining process further comprises applying a pre-determined model to at least one of: the at least one first detection signal, the at least one second detection signal, a signal representative of the background electromagnetic field, at least one topological information or subject information to determine the combined signal.

21. The apparatus as claimed in claim 1, wherein the model comprises at least one of: a convolutional neural-network or a recurrent neural network.

22. The apparatus as claimed in claim 1, wherein at least one of a) or b);
a) the signal transmitter comprises a Doppler-radar based signal transmitter; or
b) the signal receiver and signal transmitter form part of a dual-band transceiver.

23. The apparatus as claimed in claim 1, wherein
a) the first carrier frequency in the range 300 MHz to 300 GHz or, between 1 GHz and 10 GHz, and
b) the second carrier frequency is in the range 300 MHz to 300 GHz or, between 1 GHz and 10 GHz.

24. (canceled)

25. A method comprising:
transmitting at least one radar signal at a first carrier frequency and a second radar signal at a second carrier frequency
receiving at least one radar return signal;
produce at least one first detection signal for the first carrier frequency and at least one second detection signal for the second carrier frequency in response to receiving the at least one radar return signal, wherein the first carrier frequency and the second carrier frequencies are such that at least one of the at least one first detection signal or the at least one second detection signal comprise a contribution from the bio-signal and a contribution from at least one of a further bio-signal or a background;
perform a signal combining process with the at least one first detection signal and the at least one second detection signal to produce at least one combined signal comprising an at least reduced contribution from at least one of the further bio-signal or the background.

26. The apparatus of claim 1, wherein the first carrier frequency is 2.45 GHz and the second carrier frequency is 5.8 GHz.
